(19) United States
(12) Patent Application Publication
(10) Pub. No.: US 2025/0352012 A1
(43) Pub. Date: Nov. 20, 2025

(54) DOORMAT SYSTEMS FOR CLEANING AND PURIFICATION

(71) Applicant: Mario Sanchez, New York, NY (US)

(72) Inventor: Mario Sanchez, New York, NY (US)

(21) Appl. No.: 18/899,238

(22) Filed: Sep. 27, 2024

Related U.S. Application Data
(60) Provisional application No. 63/649,555, filed on May 20, 2024.

(51) Int. Cl. A47L 7/00; A47L 9/12; A47L 9/28; A47L 23/26; A61L 2/10; A61L 2/24; A61L 2/26

(52) U.S. Cl. CPC A47L 7/0061; A47L 7/0047; A47L 9/12; A47L 9/2805; A47L 9/2884; A47L 23/266; A61L 2/10; A61L 2/24; A61L 2/26; A61L 2202/11; A61L 2202/14; A61L 2202/17

(57) ABSTRACT

In one embodiment, the invention provides a UV suction door mat that comprises an air duct having an inner and outer portion. The outer portion is configured to come in contact with footwear of a user. The inner portion comprises a UV emitter that emits UV radiation having wavelength in the UV-C region. The inner portion is coupled to a suction mechanism that sucks air in order to remove debris stuck to the bottom of the footwear. The suction is achieved by a vacuum pump connected to the air duct through a hose. The hose is coupled to a container configured to collect the debris, dust, etc. from the footwear. The UV suction door mat of the invention provides the advantage of simultaneous cleaning and disinfecting the footwear. The UV suction door mat may also comprise suitable sensors to activate the UV emitter and the vacuum.

### CROSS REFERENCE TO RELATED APPLICATIONS

The present application claims priority to U.S. Provisional Patent Application Ser. No. 63/649,555 filed on May 20, 2024, incorporated herein by reference in its entirety.

### TECHNICAL FIELD

Various embodiments relate generally to cleaning and disinfecting door mats.

### BACKGROUND

Door mats are generally constructed from materials like coir, rubber, or synthetic fibers, and are configured to withstand high foot traffic. Surface textures of door mats may be coarse or ribbed in order to scrape dirt effectively. Non-slip backings on door mats ensure stability and minimize slip risks. Door mats are suitable for various entrances, including residential, office, and commercial. Door mats are easy to clean, requiring just a shake or sweep, making them a low-maintenance option for busy doorways.

UV lights that are designed for sterilization emit ultraviolet radiation to eliminate bacteria and viruses. UV lights wavelengths in the ultraviolet range are optimal for disinfection, breaking down microbial DNA. UV lights are versatile, suitable for medical facilities, laboratories, and home use. UV lights are easy to install and require minimal maintenance, making UV lights an efficient solution for sanitization.

Vacuum devices, engineered for cleaning, utilize suction to remove dirt and debris from surfaces. Vacuum devices' designs range from handheld to industrial, accommodating various cleaning needs. Vacuum devices often feature filters to trap fine particles, enhancing air quality. Vacuum devices are user-friendly, with detachable components for easy emptying and cleaning. Vacuum devices provide a practical solution for maintaining cleanliness in residential, office, and commercial spaces.

### SUMMARY

Apparatus and associated methods relate to an ultra-violet (UV) suction door mat designed for disinfecting and cleaning. In an illustrative example, a UV suction door mat of the invention includes a vacuum head, a mat, a UV emitter device, a filter, an air filtration system, and a compressor. The UV emitter device is used to disinfect air and surfaces from the door mat. The mat may be affixed to the floor and designed to be level with the floor to prevent tripping. Various embodiments may advantageously integrate a suitable sensor that activates the UV suction door mat as a user approaches, enhancing the disinfection and cleaning process as the user wipes their feet.

In various embodiments, additional components may be included in order to achieve one or more advantages. For example, some embodiments may include a brush for users to wipe their footwear on, further aiding in the removal of dirt and debris. The mat may, for example, also include a replaceable filter, ensuring long-term effectiveness and ease of maintenance. Additionally, the inclusion of a replaceable battery allows for cordless operation, increasing the mat's versatility and convenience of placement. Through these features, users may effectively clean and disinfect their footwear before entering the house, significantly reducing the transmission of germs and improving indoor air quality. Moreover, by being level with the floor, the mat may prevent tripping, ensuring safety for all users.

UV rays are known to disinfect surfaces through a mechanism known as ultraviolet germicidal irradiation, leveraging UV-C light to inactivate microorganisms by damaging their DNA or RNA, thus preventing replication. This method is particularly effective against a wide range of pathogens, including bacteria, viruses, and molds. In the context of this invention, as users wipe their footwear, germs present on the footwear surfaces are exposed to UV-C radiation, significantly reducing the microbial load. This process ensures a cleaner, more hygienic entry into indoor environments.

The vacuum component of the UV suction door mat plays a role in physical cleaning, specifically targeting the removal of dirt and debris from footwear, such as those accumulated from outdoor activities or dirty footwear. By creating a suction force, the vacuum component draws in air along with loosened dirt particles, effectively sucking in dust from footwear as they are wiped on the mat. These particles are then captured by the mat's filtration system, preventing them from re-entering the indoor environment. The integration of this vacuum feature ensures that the UV suction door mat disinfects and physically cleans footwear, enhancing the overall cleanliness and reducing the spread of dirt and germs indoors.

In some embodiments, the UV suction door mat may be integrated with a suitable computing device such as a smartphone, thus allowing users to activate the mat remotely via a suitable program or a mobile application. This feature enables users to prepare the mat for use before reaching the doorstep, ensuring that the UV disinfection and vacuum functions are already operational upon their arrival. The smartphone connectivity could also provide additional functionalities such as scheduling cleaning times, adjusting UV intensity, and monitoring filter and battery status. This integration further enhances user convenience and ensures that the mat's cleaning and disinfecting processes are as efficient and effective as possible, allowing for greater control over home sanitation practices.

Mobile applications and software programs significantly enhance the functionality and user experience of the UV suction door mat by enabling remote control and monitoring capabilities. Through a dedicated mobile app, users can activate the mat's UV disinfection and vacuum features from their smartphones, allowing for pre-emptive cleaning before entering the home. Additionally, the app may, for example, facilitate scheduling of cleaning sessions, customization of UV intensity levels, and notifications about maintenance needs such as filter replacements or battery status. This integration may, for example, provides convenience as well as allow users to maintain optimal cleanliness and hygiene effortlessly. The ability to control and monitor the mat remotely transforms it into a smart home device that aligns with modern, technology-driven lifestyles, ensuring that users can maintain a clean and healthy living environment more effectively.

The details of various embodiments are set forth in the accompanying drawings and the description below. Other features and advantages will be apparent from the description and drawings, and from the claims.

### BRIEF DESCRIPTION OF THE DRAWINGS

These and other features, aspects, and advantages of the present invention will become better understood when the following detailed description is read with reference to the accompanying drawings in which like characters represent like parts throughout the drawings.

FIG. 1 depicts an exemplary use-case scenario of an exemplary UV suction door mat employed in an illustrative use-case scenario.

FIG. 2A depicts a UV suction door mat that includes an exemplary electric motor and a battery.

FIG. 2B depicts the UV suction door mat that includes an exemplary electric motor and a battery.

FIG. 2C depicts the UV suction door mat that includes an exemplary hose connection between an electric motor and a battery with an air duct.

FIG. 2D depicts the UV suction door mat that includes an exemplary air duct with a UV light.

FIG. 2E depicts the UV suction door mat that includes an exemplary air duct with a UV light.

FIG. 2F depicts the UV suction door mat that includes an exemplary interior air pump assembly, an air filter, and a housing.

FIG. 2G depicts the UV suction door mat that includes an exemplary trigger mechanism.

FIG. 2H depicts the UV suction door mat that includes an exemplary interior air pump assembly, an air filter, and a housing.

FIG. 3 is a block diagram representation of the UV suction door mat.

### DETAILED DESCRIPTION OF ILLUSTRATIVE EMBODIMENTS

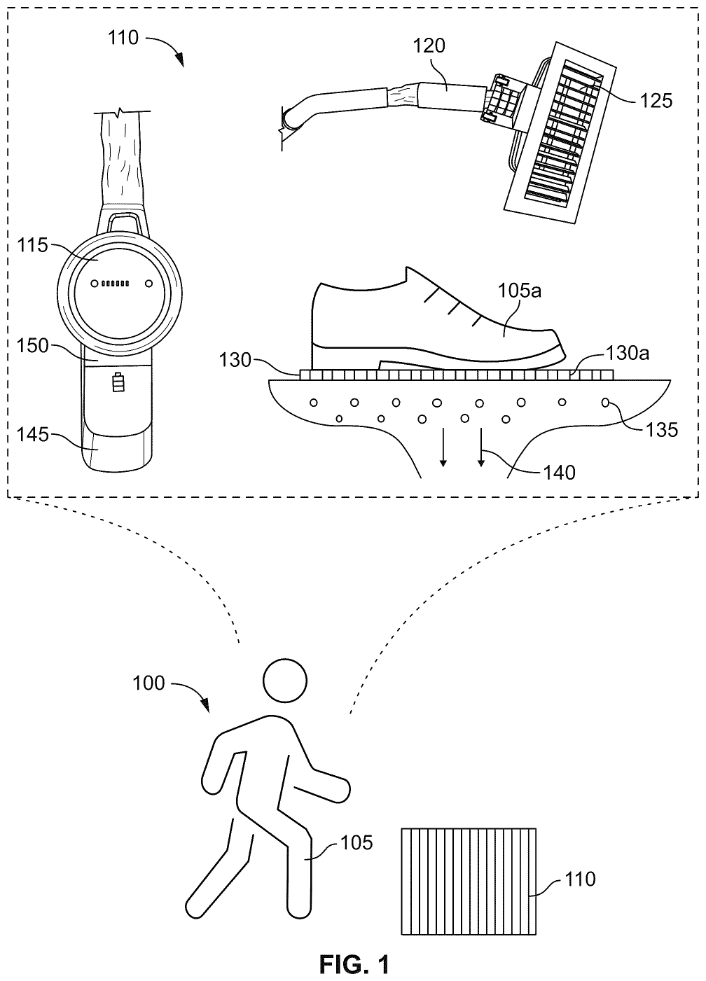

Turning to drawings, FIG. 1 depicts an exemplary use-case scenario of an exemplary UV suction door mat employed in an illustrative use-case scenario 100. The illustrative use-case scenario 100 includes a user 105 shown here wearing footwear 105a. As used herein, the term footwear is used to mean any outer covering for feet, and may include, for example, but not limited to, shoes, boots, brogues, high heels, sneakers, sandals, slippers, and the like. A UV suction door mat 110 includes an air duct 125 that comprises an outer portion configured to come into contact with the footwear and an inner portion. When the user steps onto a UV suction door mat 110, a suction mechanism made available in the inner portion of the air duct sucks air 140, thus sucking dirt particles 135 that may be stuck on the footwear 105a. The inner portion comprises a UV emitter device 130 that is configured to release UV rays 130a. The UV rays 130a cleans the footwear 105a from germs and bacteria while an air pump sucks the dirt from the footwear simultaneously. The inner portion of the air duct 125 is coupled to a hose 120, which in turn is coupled to an air pump 115 powered by a battery 145. The air pump is coupled to a container 150, which stores dirt.

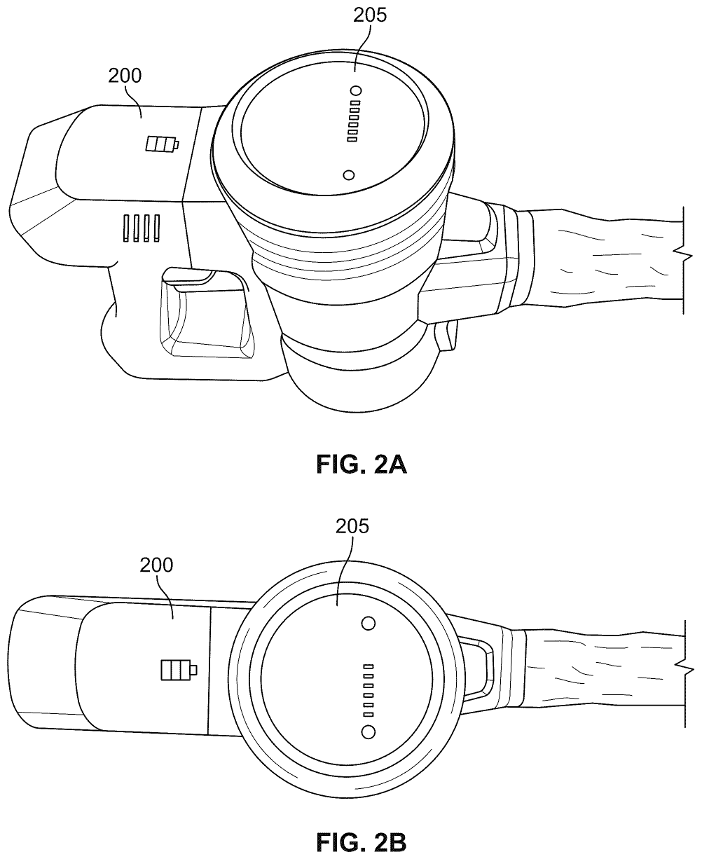

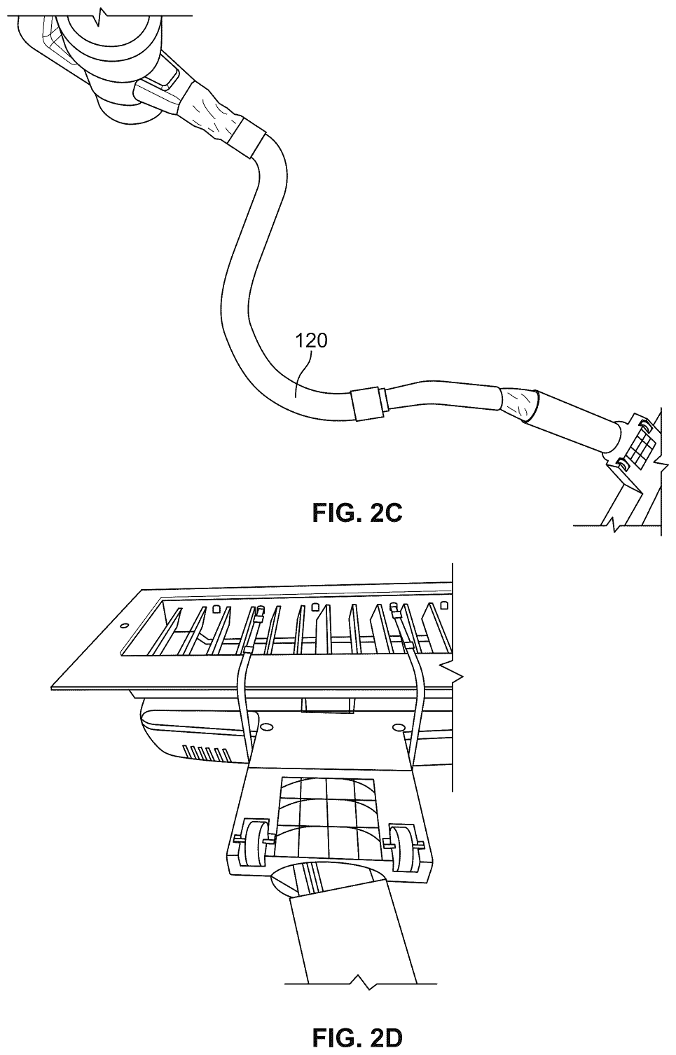

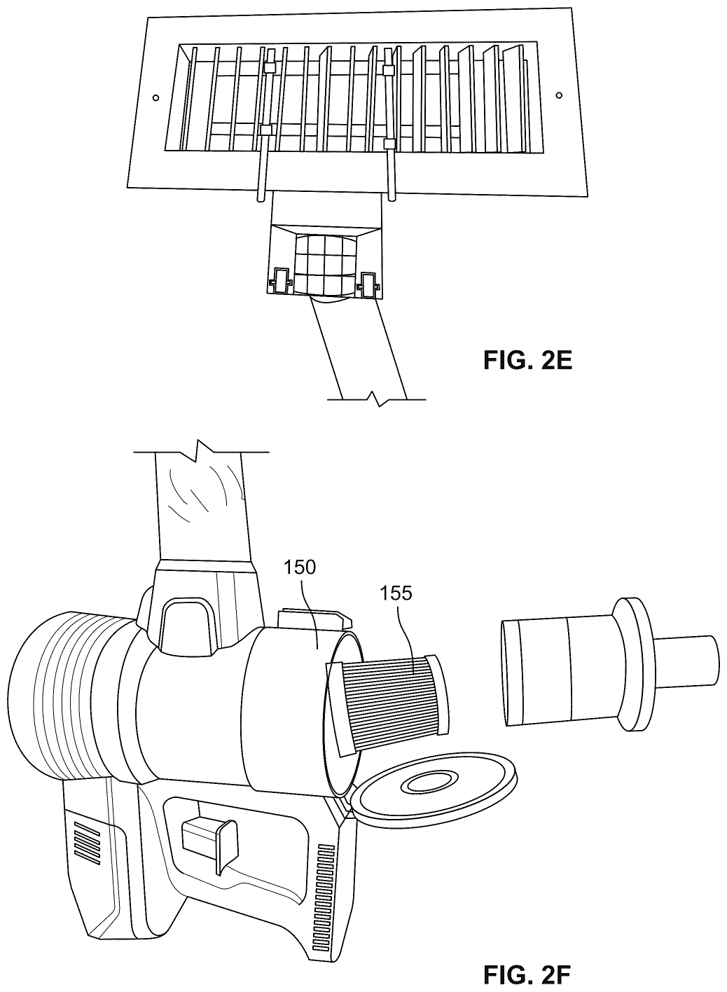

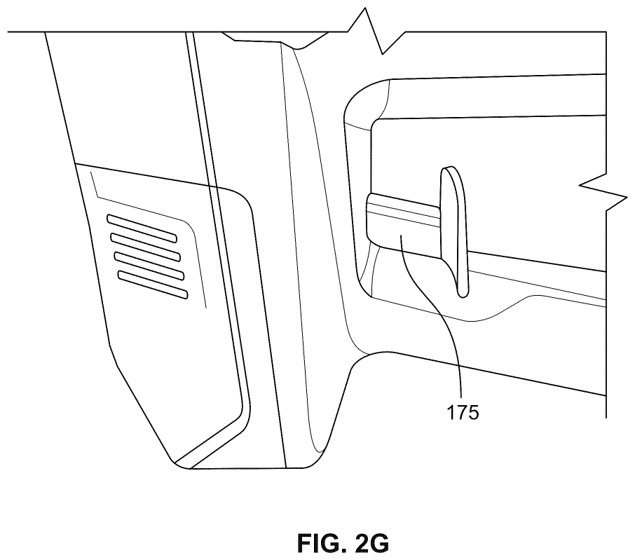

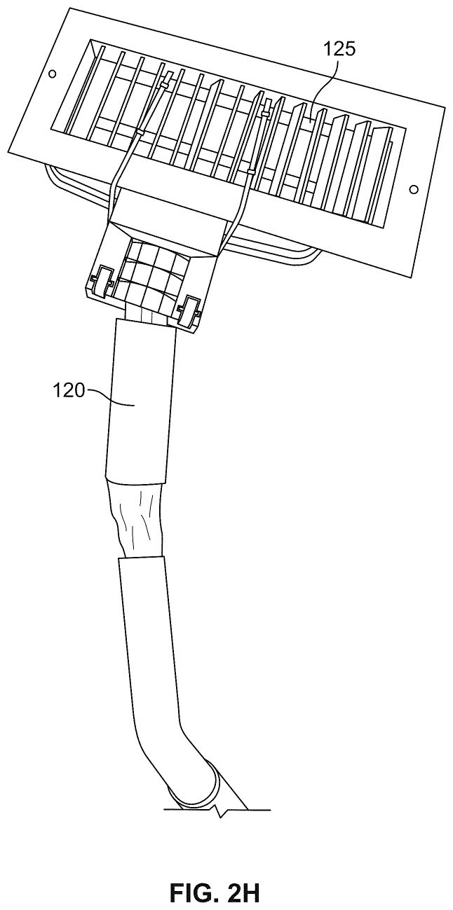

FIGS. 2A to 2H showcase the components and features of a UV suction door mat. FIGS. 2A and 2B show the device's battery 200 and motor 205 that power the UV disinfection and suction mechanisms. FIG. 2C depicts the hose 120 used for directing airflow and debris. FIGS. 2D and 2E show the UV door mat in action, highlighting its disinfectant and suction capabilities. FIG. 2F shows the container 150 with an air filter 155 for trapping contaminants (not shown). FIG. 2G shows a trigger in the form of an exemplary proximity sensor for automated activation. Lastly, FIG. 2H shows the mat in a practical use scenario, encapsulating the device's comprehensive cleaning technology.

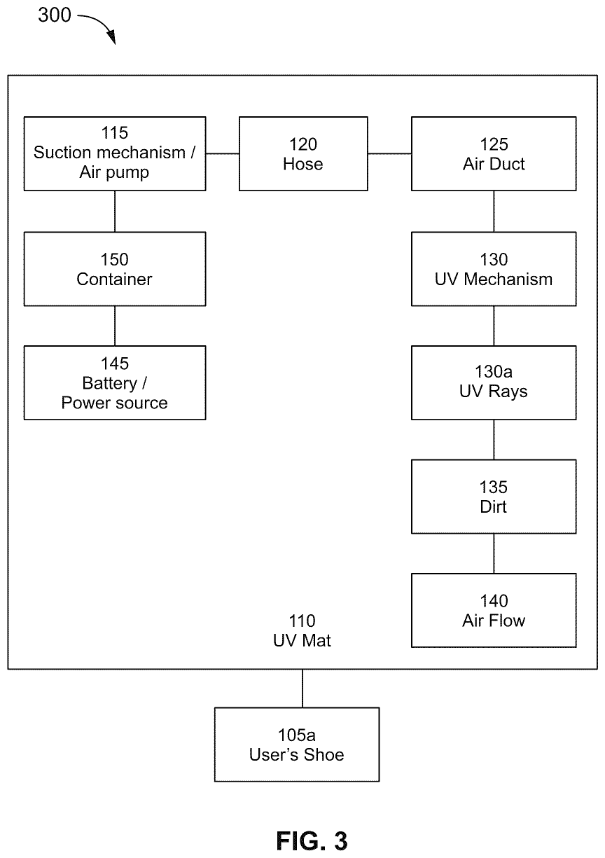

FIG. 3 is a block diagram 300 of the exemplary UV suction door mat 110. The UV suction door mat 110 includes a suction mechanism 115. The UV suction door mat 110 includes a hose 120. The hose 120 directs the movement of dirt and air specifically between the UV Mat 110 and the container 150. The UV suction door mat 110 includes an air duct 125 that directs airflow within the UV Mat 110 to enhance the suction effectiveness. The UV suction door mat 110 includes a UV mechanism 130 that emits ultraviolet light rays 130a to sanitize surfaces by neutralizing pathogens on contact. Dirt 135 represents the particulate matter removed from user's footwear by the UV Mat 110. The dirt 135 may, for example, be removed by air flow 140. Air flow 140 through the system ensures efficient movement of dirt towards the container.

In some embodiments, the UV suction door mat is designed to seamlessly integrate into a home environment, featuring a level mat surface that prevents tripping hazards. This embodiment includes a UV emitter device strategically placed to disinfect both the air and surfaces, ensuring a reduction of pathogens as users wipe their feet on the mat. Additionally, a suitable sensor such as a proximity sensor may be incorporated, activating the mat as a user approaches, which enhances the disinfection process automatically without the need for manual operation. Other sensors useful in the invention include weight sensors, optical sensors, motion sensors, and the like, and combinations thereof.

In some embodiments, the UV suction door mat includes a series of user-friendly features that promote hygiene and ease of use. For example, the outer portion of the air duct is equipped with a brush that facilitates effective removal of dirt and debris, especially sticky materials such as clay or mud, from their footwear. It may also include a replaceable filter and a replaceable battery, enabling cordless operation and easy maintenance. These features collectively ensure that users can clean and disinfect their footwear effectively before entering their homes, thereby reducing the transmission of germs and improving indoor air quality.

The functionality of the UV suction door mat is augmented by a vacuum component that enhances physical cleaning capabilities. This component creates a suction force that draws in air and loosens dirt particles from footwear during the wiping process. Dirt and debris, such as those picked up from outdoor activities, are efficiently sucked into the mat and captured by its filtration system. This integration of the vacuum feature not only disinfects but also physically cleans the footwear, helping to maintain indoor cleanliness and reduce the spread of dirt and pathogens.

In some embodiments, the UV suction door mat utilizes an advanced air filtration system that works in tandem with the UV emitter to enhance the decontamination process. This system can capture finer particulate matter, ensuring that the air and surfaces are not only free from visible dirt but also from microscopic pathogens. Such a configuration is particularly beneficial in environments where air quality and surface cleanliness are paramount.

In some embodiments, the UV suction door mat could feature adjustable UV intensity levels that can be customized according to different sanitation needs. This flexibility allows users to select more intense disinfection for heavily soiled footwear or a milder setting for regular sanitization, accommodating a wide range of use scenarios and user preferences.

Although various embodiments have been described with reference to the figures, other embodiments are possible. Although an exemplary system has been described with reference to FIG. 1-3, other implementations may be deployed in other industrial, scientific, medical, commercial, and/or residential applications.

In industrial settings, UV technology and vacuum systems may, for example, be used for maintaining hygiene and safety standards. For example, UV light is instrumental in water treatment processes, efficiently disinfecting large volumes of water without chemical additives, thus preventing potential chemical contamination. Vacuum systems may, for example, play a role in dust collection and air purification, especially in factories and warehouses where maintaining air quality is an important parameter to prevent respiratory issues and ensure a clean working environment. UV door mats may, for example, be used to satisfy these industrial environments.

In scientific research, UV technology may, for example, be used to sterilize laboratory equipment and workspaces, ensuring experiments are conducted in aseptically maintained environments. This may, for example, be important in biological research where contamination can skew results. Additionally, vacuum technology aids may, for example, be used to maintain controlled environments in experiments that require specific atmospheric conditions, demonstrating its versatility beyond mere cleaning applications. UV door mats may, for example, be used to satisfy these scientific environments.

The medical sector may, for example, leverage UV technology extensively for disinfecting surgical rooms, medical instruments, and even the air, significantly reducing the risk of infections. Vacuum systems may, for example, be integrated into medical devices for various purposes, including respiratory therapy equipment and surgical instruments, highlighting their importance in patient care and treatment efficacy. UV door mats may, for example, be used to satisfy these medical environments.

Commercially, UV systems may, for example, be utilized for sterilizing surfaces in food processing and packaging, extending the shelf life of products while maintaining food safety. UV systems may, for example, be installed into airports (e.g., on plane, through security), and/or on public transport. Vacuum systems may, for example, be used in commercial settings, for cleaning purposes in hotels, office buildings, and retail spaces to ensure a clean and inviting environment for customers and employees alike. UV door mats may, for example, be used to satisfy these commercial environments.

In residential environments, UV air purifiers may, for example, be used to neutralize airborne pathogens, enhancing indoor air quality and protecting inhabitants' health. Vacuum systems equipped with HEPA filters may, for example, are favored for their effectiveness in removing allergens and pollutants from home environments, contributing to cleaner, healthier living spaces. UV door mats may, for example, be used to satisfy these residential environments.

Temporary auxiliary energy inputs may be received, for example, from chargeable or single use batteries, which may enable use in portable or remote applications. Some embodiments may operate with other DC voltage sources, such as a 9V (nominal) batteries, for example. Alternating current (AC) inputs, which may be provided, for example from a 50/60 Hz power port, or from a portable electric generator, may be received via a rectifier and appropriate scaling. Provision for AC (e.g., sine wave, square wave, triangular wave) inputs may include a line frequency transformer to provide voltage step-up, voltage step-down, and/or isolation.

In various embodiments, some bypass circuits implementations may be controlled in response to signals from analog or digital components, which may be discrete, integrated, or a combination of each. Some embodiments may include programmed, programmable devices, or some combination thereof (e.g., PLAs, PLDs, ASICs, microcontroller, microprocessor), and may include one or more data stores (e.g., cell, register, block, page) that provide single or multi-level digital data storage capability, and which may be volatile, non-volatile, or some combination thereof. Some control functions may be implemented in hardware, software, firmware, or a combination of any of them.

Computer program products may contain a set of instructions that, when executed by a processor device, cause the processor to perform prescribed functions. These functions may be performed in conjunction with controlled devices in operable communication with the processor. Computer program products, which may include software, may be stored in a data store tangibly embedded on a storage medium, such as an electronic, magnetic, or rotating storage device, and may be fixed or removable (e.g., hard disk, floppy disk, thumb drive, CD, DVD).

Although an example of a system, which may be portable, has been described with reference to the above figures, other implementations may be deployed in other processing applications, such as desktop and networked environments.

Although particular features of an architecture have been described, other features may be incorporated to improve performance. For example, caching (e.g., L1, L2, . . . ) techniques may be used. Random access memory may be included, for example, to provide scratch pad memory and/or to load executable code or parameter information stored for use during runtime operations. Other hardware and software may be provided to perform operations, such as network or other communications using one or more protocols, wireless (e.g., infrared) communications, stored operational energy and power supplies (e.g., batteries), switching and/or linear power supply circuits, software maintenance (e.g., self-test, upgrades), and the like. One or more communication interfaces may be provided in support of data storage and related operations.

Some systems may be implemented as a computer system that can be used with various implementations. For example, various implementations may include digital circuitry, analog circuitry, computer hardware, firmware, software, or combinations thereof. Apparatus can be implemented in a computer program product tangibly embodied in an information carrier, e.g., in a machine-readable storage device, for execution by a programmable processor; and methods can be performed by a programmable processor executing a program of instructions to perform functions of various embodiments by operating on input data and generating an output. Various embodiments can be implemented advantageously in one or more computer programs that are executable on a programmable system including at least one programmable processor coupled to receive data and instructions from, and to transmit data and instructions to, a data storage system, at least one input device, and/or at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, which may include a single processor or one of multiple processors of any kind of computer. Generally, a processor will receive instructions and data from a read-only memory or a random-access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including, by way of example, semiconductor memory devices, such as EPROM, EEPROM, and flash memory devices; magnetic disks, such as internal hard disks and removable disks; magneto-optical disks; and CD-ROM and DVD-ROM disks. The processor and the memory can be supplemented by, or incorporated in, ASICs (application-specific integrated circuits).

In some implementations, each system may be programmed with the same or similar information and/or initialized with substantially identical information stored in volatile and/or non-volatile memory. For example, one data interface may be configured to perform auto configuration, auto download, and/or auto update functions when coupled to an appropriate host device, such as a desktop computer or a server.

In some implementations, one or more user-interface features may be custom configured to perform specific functions. Various embodiments may be implemented in a computer system that includes a graphical user interface and/or an Internet browser. To provide for interaction with a user, some implementations may be implemented on a computer having a display device. The display device may, for example, include an LED (light-emitting diode) display. In some implementations, a display device may, for example, include a CRT (cathode ray tube). In some implementations, a display device may include, for example, an LCD (liquid crystal display). A display device (e.g., monitor) may, for example, be used for displaying information to the user. Some implementations may, for example, include a keyboard and/or pointing device (e.g., mouse, trackpad, trackball, joystick), such as by which the user can provide input to the computer.

In various implementations, the system may communicate using suitable communication methods, equipment, and techniques. For example, the system may communicate with compatible devices (e.g., devices capable of transferring data to and/or from the system) using point-to-point communication in which a message is transported directly from a source to a receiver over a dedicated physical link (e.g., fiber optic link, point-to-point wiring, daisy-chain). The components of the system may exchange information by any form or medium of analog or digital data communication, including packet-based messages on a communication network. Examples of communication networks include, e.g., a LAN (local area network), a WAN (wide area network), MAN (metropolitan area network), wireless and/or optical networks, the computers and networks forming the Internet, or some combination thereof. Other implementations may transport messages by broadcasting to all or substantially all devices that are coupled together by a communication network, for example, by using omni-directional radio frequency (RF) signals. Still other implementations may transport messages characterized by high directivity, such as RF signals transmitted using directional (i.e., narrow beam) antennas or infrared signals that may optionally be used with focusing optics. Still other implementations are possible using appropriate interfaces and protocols such as, by way of example and not intended to be limiting, USB 2.0, Firewire, ATA/IDE, RS-232, RS-422, RS-485, 802.11 a/b/g, Wi-Fi, Ethernet, IrDA, FDDI (fiber distributed data interface), token-ring networks, multiplexing techniques based on frequency, time, or code division, or some combination thereof. Some implementations may optionally incorporate features such as error checking and correction (ECC) for data integrity, or security measures, such as encryption (e.g., WEP) and password protection.

In various embodiments, the computer system may include Internet of Things (IoT) devices. IoT devices may include objects embedded with electronics, software, sensors, actuators, and network connectivity which enable these objects to collect and exchange data. IoT devices may be in-use with wired or wireless devices by sending data through an interface to another device. IoT devices may collect useful data and then autonomously flow the data between other devices.

Various examples of modules may be implemented using circuitry, including various electronic hardware. By way of example and not limitation, the hardware may include transistors, resistors, capacitors, switches, integrated circuits, other modules, or some combination thereof. In various examples, the modules may include analog logic, digital logic, discrete components, traces and/or memory circuits fabricated on a silicon substrate including various integrated circuits (e.g., FPGAs, ASICs), or some combination thereof. In some embodiments, the module(s) may involve execution of preprogrammed instructions, software executed by a processor, or some combination thereof. For example, various modules may involve both hardware and software.

A number of implementations have been described. Nevertheless, it will be understood that various modifications may be made. For example, advantageous results may be achieved if the steps of the disclosed techniques were performed in a different sequence, or if components of the disclosed systems were combined in a different manner, or if the components were supplemented with other components. Accordingly, other implementations are contemplated.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A UV suction door mat that comprises:
an air duct having an outer portion configured to come into contact with a footwear and an inner portion;
the inner portion of the air duct comprises an UV emitter;
the inner portion air duct coupled to a hose;
the hose coupled to an air pump powered by a power source; and
the air pump coupled to a container.

2. The UV suction door mat of claim 1 wherein the power source is a rechargeable battery.

3. The UV suction door mat of claim 1 wherein the container comprises a filter.

4. The UV suction door mat of claim 1 wherein the UV emitter is configured to emit UV light having a wavelength in the range of from about 180 nanometers to about 280 nanometers.

5. The UV suction door mat of claim 1 wherein the UV emitter is configured to emit UV light having a wavelength in the range of from about 200 nanometers to about 240 nanometers.

6. The UV suction door mat of claim further comprising a sensor.

7. The UV suction door mat of claim 6 wherein the sensor is at least one of a weight sensor, optical sensor, motion sensor, proximity sensor, or combinations thereof.

8. The UV suction door mat of claim 1 wherein the outer portion of the air duct comprises a brush configured to come in contact with the footwear.

9. The UV suction door mat of claim 2 further comprising battery status sensor.

10. The UV suction door mat of claim 3 further comprising a filter sensor.

11. The UV suction door mat of claim 1 wherein the UV emitter is configured to emit UV radiation at an intensity that is user controlled.

12. The UV suction door mat of claim 1 wherein the UV suction door mat is monitored, controlled, or both from a computing device.

13. The UV suction door mat of claim 12 wherein the computing device is at least one of a computer, a smartphone, a tablet, or combinations thereof.

14. The UV suction door mat of claim 12 wherein the monitoring, controlling or both is through at least one of a mobile application, a software program, or combinations thereof.
